SEQUENCE LISTING (10 sequences)

SEQ ID NO: 1 - DNA, 21 bp, Artificial Sequence
  RT-PCR fw TSPAN8 primer
gctgcatgcttctgttgtttt

SEQ ID NO: 2 - DNA, 20 bp, Artificial Sequence
  rev TSPAN8 primer
aacacaattatggcttcctg

SEQ ID NO: 3 - DNA, 20 bp, Artificial Sequence
  fw COL1A2 primer
gtggttactactggattgac

SEQ ID NO: 4 - DNA, 20 bp, Artificial Sequence
  rev COL1A2 primer
ctgccagcattgatagtttc

SEQ ID NO: 5 - DNA, 20 bp, Artificial Sequence
  fw LGALS4 primer
ttaccctggtcccggacatt

SEQ ID NO: 6 - DNA, 20 bp, Artificial Sequence
  rev LGALS4 primer
agcctcccgaaatatggcac

SEQ ID NO: 7 - DNA, 21 bp, Artificial Sequence
  fw CEACAM6 primer
cacagtctctggaagtgctcc

SEQ ID NO: 8 - DNA, 18 bp, Artificial Sequence
  rev CEACAM6 primer
ggccagcactccaatcgt

SEQ ID NO: 9 - DNA, 21 bp, Artificial Sequence
  fw B2M primer
tgcctgccgtgtgaaccatgt

SEQ ID NO: 10 - DNA, 24 bp, Artificial Sequence
  rev B2M primer
tgcggcatcttcaaacctccatga

(19) United States
(12) Patent Application Publication
(10) Pub. No.: US 2019/0218617 A1
(43) Pub. Date: Jul. 18, 2019

(54) PROGNOSTIC METHOD

(71) Applicant: Alma Mater Studiorum - Università di Bologna, BOLOGNA (IT)

(72) Inventors: Rossella SOLMI, Bologna (IT); Maria Teresa RODIA, Salerno (IT); Mattia LAURIOLA, Bologna (IT); Gabriella MATTEI, Bologna (IT); Giampaolo UGOLINI, Bologna (IT); Isacco MONTRONI, Imola (Bologna) (IT)

(73) Assignee: Alma Mater Studiorum - Università di Bologna, BOLOGNA (IT)

(21) Appl. No.: 16/216,750

(22) Filed: Dec. 11, 2018

(30) Foreign Application Priority Data
Jan. 12, 2018 (IT) 102018000000827

(51) Int. Cl. C12Q 1/6886; C12Q 1/6851

(52) U.S. Cl. CPC C12Q 1/6886; C12Q 2600/118; C12Q 1/6851

(57) ABSTRACT

The present invention relates to a diagnostic/prognostic method for the identification of patients at low risk and high risk of developing colorectal cancer from a sample of human entire blood and/or blood fractions comprising nucleic acids, and to the use of a kit for performing said method.

### CROSS-REFERENCE TO RELATED APPLICATIONS

This application claims the priority benefit of Italian Application No. 102018000000827, filed on Jan. 12, 2018, the contents of which is hereby incorporated by reference in its entirety.

### REFERENCE TO SEQUENCE LISTING SUBMITTED ELECTRONICALLY VIA EFS-WEB

The content of the electronically submitted sequence listing (Name: BX3653RSeqListing.txt; Size: 2,089 bytes; and Date of Preparation: Dec. 11, 2018) is herein incorporated by reference in its entirety.

### DESCRIPTION

The present invention relates to a diagnostic/prognostic method for the identification of patients at low risk and high risk of developing colorectal cancer from a sample of human entire blood and/or blood fractions comprising nucleic acids, and to the use of a kit for performing said method.

### PRIOR ART

Colorectal cancer (CRC) is the third most common cancer worldwide, with almost 1.4 million new cases diagnosed in 2012. A significant survival rate is obtained if the primary tumour is detected at an early stage.

In most CRC cases, a multi-stage process develops, starting with benign pre-cancerous adenomas, which develop into aggressive metastatic carcinoma. This makes early diagnosis fundamental in order to benefit from the chances of a positive outcome for CRC patients.

Various non-invasive screening methods have been studied, including faecal tests detecting the presence of haemoglobin or blood in the faeces, and improved faecal tests which also envisage integral DNA extraction. The research of markers as a screening instrument in a patient's blood represents a research topic for colorectal cancer early diagnosis. Numerous reports include encoding mRNAs, microRNAs (miRNAs), proteins, metabolites, DNA mutations, and methylation markers. To date, the main trends of research on candidate mRNA markers generally involve various types of experimental tests: circulating tumour cells (CTCs), cancer stem cells (CSCs) (17), and circulating free RNA (cfRNA). Metastatic diffusion occurs quite early in tumour development; therefore, a specific and sensitive detection of CTCs have become crucial for diagnosis. Quantitative PCR (qPCR) has recently been described as a good method for CTC quantification.

The prior art has identified the need for a simple and reliable test that can be performed on whole blood and that therefore does not require manipulation of faeces or extraction procedures for CTCs or CSCs or blood fractions. Amongst other things, a reliable test that can be performed on whole blood does not entail the risk of CTC and/or CSC loss over the various handling steps, the number of these cells being critical for the assay success.

RNA analysis is based on the fact that tumour phenotype variations are associated with changes in the mRNA levels of genes regulating or influencing these variations. This has led to the use of qRT-PCR.

The authors of the present invention described a diagnostic method and kit for the early diagnosis of colorectal cancer in patent application IT 102015000016638 and in the corresponding application PCT WO2016/185451.

In particular, the above-mentioned patent applications describe a method for diagnosing colorectal cancer on the basis of a sample of human whole blood and/or blood fractions containing nucleic acids, said method comprising the steps of

a. extracting total RNA or mRNA from said sample

b. carrying out a quantitative analysis of the human genes TSPAN8, LGALS4, CEACAM6, COL1A2 mRNAs, in which

an overexpression of TSPAN8 and COL1A2 and the underexpression of LGALS4 and CEACAM6 compared to reference values indicate the presence of colorectal cancer.

Colorectal cancer develops, in most cases, from what are known as adenomatous polyps, which are lesions caused by abnormal cell growth, but are initially benign and only have the capacity to develop into cancer over time.

Such lesions can currently be diagnosed only by means of colonoscopy.

Colonoscopy makes it possible, in fact, to define histologically the type of lesions present in the colon and to determine whether such lesions are low-risk and therefore will not develop into cancer, or if they are high-risk lesions and therefore have an increased likelihood of becoming cancerous lesions.

To date, there are no rapid non-invasive tests which make it possible to differentiate between low-risk and high-risk lesions and which therefore provide prognostic information regarding the health of the patient.

### SUMMARY OF THE INVENTION

The present invention provides a diagnostic/prognostic method which, in addition to providing an early diagnosis of colorectal cancer, makes it possible to distinguish between patients not suffering from colorectal cancer or high-risk lesions (that is to say lesions very likely to develop into cancer), patients who have low-risk lesions in the colon (that is to say lesions that will not develop into cancer), and also patients who, in spite of positive results of performed faecal tests, are not currently affected by high-risk tumours in the colon or by high-risk lesions.

As mentioned above, in the present state of the art, only a colonoscopy is able to allow a doctor to know whether the examined patient has high-risk colorectal lesions or tumours or whether the patient has colon lesions, but these lesions are low-risk and will not develop into colorectal cancer.

It is clear that knowing whether the patient has colorectal lesions and defining the type of lesions constitutes information essential for the doctor insofar as it allows him:

a. to have prognostic information for the patient,

b. to establish, very early on, the type of therapeutic approach to be adopted by the patient.

The authors of the present invention have surprisingly found that quantitative analysis of the markers TSPAN8, LGALS4, CEACAM6, COL1A2 makes it possible to identify patients not affected by colorectal tumours but who have low-risk lesions. The authors of the present invention have therefore developed a diagnostic/prognostic method for the identification of patients at low risk and high risk of developing colorectal cancer from a sample of human entire blood and/or blood fractions comprising nucleic acids, comprising the steps of:

a. extracting total RNA or mRNA from said sample

b. carrying out a quantitative analysis of the human genes TSPAN8, LGALS4, CEACAM6, COL1A2 mRNAs,

c. quantifying the mRNA of one more human constitutive (housekeeping) genes and carrying out a calculation of ΔCt normalised with respect to said one or more genes for each marker,

d. assessing the risk that the subject of the analysed sample has to develop colorectal cancer, wherein

for a value of ΔCt 13.6±1.2; for marker CEACAM6; a value of ΔCt 15.3±0.8 for marker LGALS4; a value of ΔCt 9.9±1.4 for marker TSPAN8; and a value of ΔCt 9.7±1.4 for marker COL1A2 the subject analysed is defined as a patient with lesions at low risk of developing colorectal cancer and,

for a value of ΔCt: 9.6±1.9 for marker TSPAN8; a value of ΔCt 9.6±2 for marker COL1A3; a value of ΔCt 14.7±1.3 for marker LGALS4 and a value of ΔCt 13.3±1.2 for marker CEACAM6 the subject analysed is defined as a patient with lesions at high risk of developing colorectal cancer or affected by CRC.

This method thus makes it possible to divide the patient population on the basis of their prognosis of developing colorectal cancer and to provide such patients with a suitable regime of prevention and therapy.

The invention also relates to the use of a kit for the diagnosis of colorectal cancer from a sample of human entire blood and/or blood fractions comprising nucleic acids, comprising reagents for the quantitative analysis of the expression of human genes TSPAN8, LGALS4, CEACAM6, COL1A2 for the identification of patients at low risk and at high risk of developing colorectal cancer.

Lastly, the invention relates to a therapeutic method for the treatment of patients at high risk of or affected by CRC, in which patients are subjected to a propaedeutic prognostic analysis for identifying if they are at low risk or at high risk of developing colorectal cancer from a sample of human entire blood and/or blood fractions comprising nucleic acids, comprising the steps of:

a. extracting total RNA or mRNA from said sample

b. carrying out a quantitative analysis of the human genes TSPAN8, LGALS4, CEACAM6, COL1A2 mRNAs,

c. quantifying, in the same sample, the mRNA of one more human constitutive (housekeeping) genes and carrying out a calculation of ΔCt normalised with respect to said one or more genes for each marker,

d. assessing the risk that the subject of the analysed sample has to develop colorectal cancer, wherein

for a value of ΔCt 13.6±1.2; for marker CEACAM6; a value of ΔCt 15.3±0.8 for marker LGALS4; a value of ΔCt 9.9±1.4 for marker TSPAN8; and a value of ΔCt 9.7±1.4 for marker COL1A2 the subject analysed is defined as a patient with lesions at low risk of developing colorectal cancer and,

for a value of ΔCt: 9.6±1.9 for marker TSPAN8; a value of ΔCt 9.6±2 for marker COL1A3; a value of ΔCt 14.7±1.3 for marker LGALS4 and a value of ΔCt 13.3±1.2 for marker CEACAM6 the subject analysed is defined as a patient with lesions at high risk of developing colorectal cancer or affected by CRC, and in which

the patients at low risk of developing colorectal cancer are subjected to long-term routine checks whilst the patients at high risk of developing colorectal cancer are subjected to surgical intervention in order to remove the lesions present in the colorectal tract and are subjected to ongoing short-term checks and optionally to therapy with anti-cancer drugs.

### DETAILED DESCRIPTION OF THE DRAWINGS

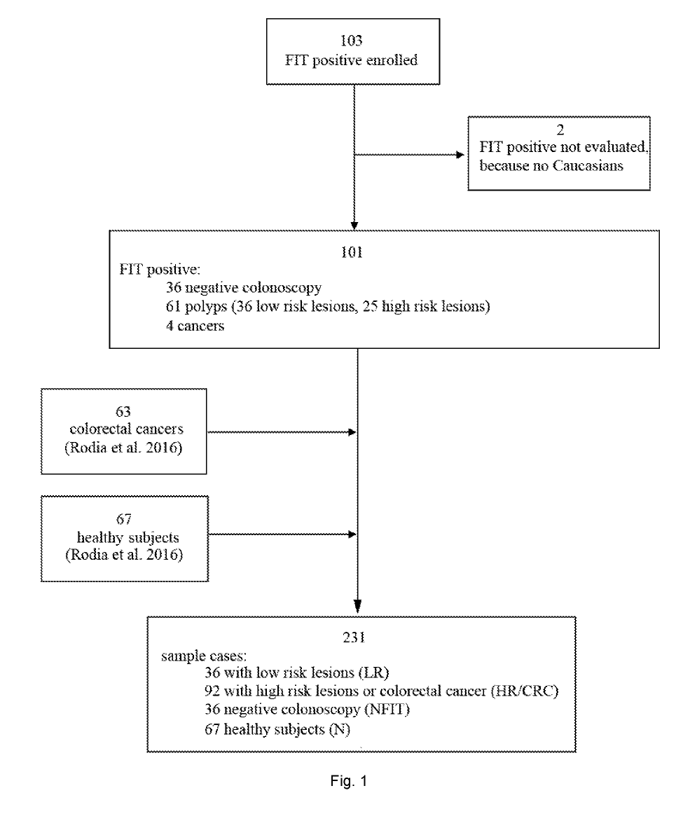

FIG. 1 The study included 101 subjects having positive results in the faecal immunochemical test (FIT test) and subjected to colonoscopy.

63 cases of colorectal carcinoma and 67 healthy reference subjects were also added, obtained from the previous study (Systematic large-scale meta-analysis identifies a panel of two mRNAs as blood biomarkers for colorectal cancer detection. Rodia M T, Ugolini G, Mattei G, Montroni I, Zattoni D, Ghignone F, Veronese G, Marisi G, Lauriola M, Strippoli P, Solmi R. Oncotarget. 2016 May 24; 7(21):30295-306.).

The colonoscopy performed on positive FIT cases made it possible to subdivide the cases (n=231) into 4 groups:

LR=subjects with low-risk lesions (n=36)

HR/CRC=subjects with high-risk lesions or CRC (n=92)

NFIT=subjects negative in the colonoscopy, but positive in the FIT (n=36)

N=healthy subjects (n=67)

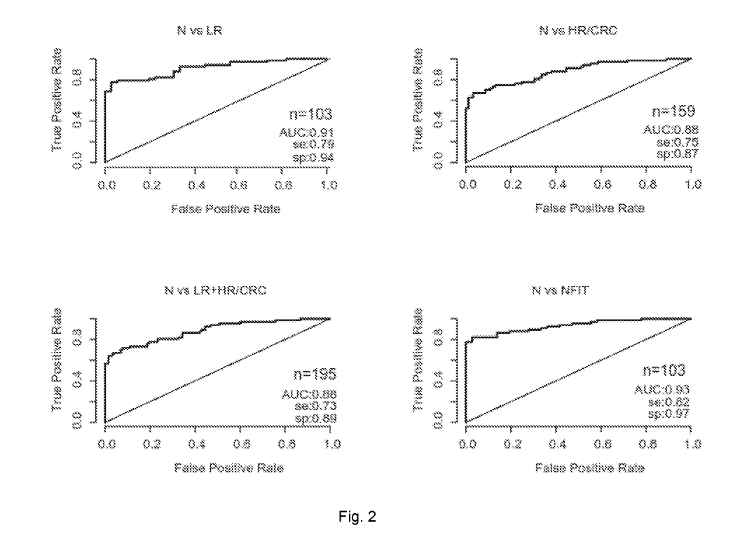

FIG. 2 ROC (Receiving Operating Characteristic) curves for the panel of markers TSPAN8, LGALS4, CEACAM6 and COL1A2.

The ROC curve represents an area (AUC) whose best value is 1 (area of a perfect square, with ordinate and abscissa coinciding with a value 100).

Climbing up from the area, and more precisely from the topmost, leftwise-projecting point (the area at issue is more or less “stepped” on the left side of the graph) the specificity percentage, sp, (on the abscissa) and the sensitivity percentage, se, (on the ordinate) are determined. The more the area tends to a square with value 1, the highest are the specificity and sensitivity percentages. The two values hardly go in the same direction, for instance, high specificity (ability to correctly identify healthy subjects) values are often related to low sensitivity (ability to correctly identify diseased subjects) values.
True positive rate=ability to correctly identify diseased subjects
False positive rate=ability to correctly identify healthy subjects
Graph in the top left corner
Comparison between healthy subjects (N) and subjects with low-risk lesions (LR)
Graph in the top right corner
Comparison between healthy subjects (N) and subjects with high-risk lesions or with colorectal carcinoma (HR/CRC).
Graph in the bottom left corner
Comparison between healthy subjects (N) and subjects with low-risk lesions
(LR) and subjects with high-risk lesions or with colorectal carcinoma (HR/CRC).
Graph in the bottom right corner
Comparison between healthy subjects (N) and subjects with negative colonoscopy results and positive FIT results (NFIT).

### DETAILED DESCRIPTION OF THE SEQUENCES

RT-PCR forward primer for TSPAN8
SEQ ID NO: 1
gctgcatgcttctgttgtttt
RT-PCR reverse primer for TSPAN8
SEQ ID NO: 2
aacacaattatggcttcctg
RT-PCR forward primer for COL1A2
SEQ ID NO: 3
gtggttactactggattgac
RT-PCR reverse primer for COL1A2
SEQ ID NO: 4
ctgccagcattgatagtttc
RT-PCR forward primer for LGALS4
SEQ ID NO: 5
ttaccctggtcccggacatt
RT-PCR reverse primer for LGALS4
SEQ ID NO: 6
agcctcccgaaatatggcac
RT-PCR forward primer for CEACAM6
SEQ ID NO: 7
cacagtctctggaagtgctcc
RT-PCR reverse primer for CEACAM6
SEQ ID NO: 8
Ggccagcactccaatcgt
RT-PCR forward primer for B2M
SEQ ID NO: 9
tgcctgccgtgtgaaccatgt
RT-PCR reverse primer for B2M
SEQ ID NO: 10
tgcggcatcttcaaacctccatga

### Definitions

For the purposes of the present description, in accordance with the literature, patients at high risk of developing CRC are defined as those that have from 5 to more than 5 adenomas or 1 adenoma equal to or larger than 20 mm. The lesions are removed and the patients are subjected to colonoscopy within a year following the removal of the lesions. Patients at low risk of developing CRC are defined as those having tubular adenomas with dysplasia of small dimensions <10 mm, totaling 1-2 in number. These patients continue to be monitored by means of the FIT test every 2 years.

As per the literature, high-risk lesions of the colorectal tract (with progression similar to CRC) are understood to be lesions with a size at least equal to or greater than 10 mm and/or lesions with morphology of the adenoma type or of the serrated sessile type, whereas low-risk lesions of the colorectal tract (with progression similar to CRC) are understood to be lesions all having a size less than 10 mm, with low-level dysplasia (and therefore neither of the adenoma type or the serrated sessile type).

### DETAILED DESCRIPTION OF THE INVENTION

As discussed in the section related to the prior art, colorectal cancer is a disease with a very high incidence, for which it is necessary to have the availability of diagnostic screening methods that are reliable and easy to carry out. In particular, it is important for prognostic purposes and in order to establish the appropriate therapies to have quick and reliable methods for distinguishing the various classes of patients at risk of developing colorectal cancer.

For diagnostic and prognostic purposes, in addition, the higher the sensitivity and the specificity, the more reliable the diagnosis.

The sensitivity and specificity values are obtained from a graph (ROC curve) representing an area (AUC) whose best value is 1 (area of a perfect square with ordinate and abscissa coinciding with value 100).

Climbing up from the area, and more precisely from the topmost, leftwise-projecting point (the area at issue is more or less “stepped” on the left side of the graph) the specificity percentage (on the abscissa) and the sensitivity percentage (on the ordinate) are determined. The more the area tends to a square with value 1, the higher are the specificity and sensitivity percentages.

However, as already discussed above, the sensitivity and specificity values hardly go in the same direction; often, in fact, high specificity (ability to correctly identify healthy subjects) values are related with low sensitivity (ability to correctly identify diseased subjects) values.

For instance, the Faecal Occult Blood Test (FOBT) commonly used for colorectal cancer diagnosis, though being highly specific (in fact, it is able to identify even very small blood traces present in the faeces), is not very sensitive, as it does not enable to discriminate the cases in which bleeding occurs due to reasons independent from the presence of a tumour, so much that in a subsequent colonoscopy inspection only ⅓ of FOBT-positive cases are found to be diseased.

Evidently, therefore, a test with high specificity and sensitivity of not only diagnostic, but also prognostic value is needed in the present state of the art, since a test meeting these requirements has an economical value added (as it enables to proceed with a colonoscopy only on individuals actually having a very high probability of having a colorectal cancer), but, above all, an enormous psychological value, as it enables to rule out false positives with higher accuracy, avoiding the entailed psychological repercussions on the same individuals.

The authors of the present invention have surprisingly found that a diagnostic method previously developed by them and published in patent application IT 102015000016638 and in the corresponding application PCT WO2016/185451, adapted as appropriate, makes it possible to identify, in the patient population previously identified as simply not affected by CRC, a subpopulation of patients with lesions at low risk of developing CRC.

The present invention therefore provides a diagnostic/prognostic method for the identification of patients at low risk and high risk of developing colorectal cancer from a sample of human entire blood and/or blood fractions comprising nucleic acids, comprising the steps of:

a. extracting total RNA or mRNA from said sample

b. carrying out a quantitative analysis of the human genes TSPAN8, LGALS4, CEACAM6, COL1A2 mRNAs,

c. quantifying, in the same sample, the mRNA of one more human constitutive (housekeeping) genes and carrying out a calculation of ΔCt normalised with respect to said one or more genes for each marker,

d. assessing the risk that the subject of the analysed sample has to develop colorectal cancer, wherein

for a value of ΔCt 13.6±1.2; for marker CEACAM6; a value of ΔCt 15.3±0.8 for marker LGALS4; a value of ΔCt 9.9±1.4 for marker TSPAN8; and a value of ΔCt 9.7±1.4 for marker COL1A2 the subject analysed is defined as a patient with lesions at low risk of developing colorectal cancer and,

for a value of ΔCt: 9.6±1.9 for marker TSPAN8; a value of ΔCt 9.6±2 for marker COL1A3; a value of ΔCt 14.7±1.3 for marker LGALS4 and a value of ΔCt 13.3±1.2 for marker CEACAM6 the subject analysed is defined as a patient with lesions at high risk of developing colorectal cancer or affected by CRC.

According to the invention, the human gene TSPAN8 is Tetraspanin 8 gene, whose RNA has RNA GenBank accession number NM 004616.

According to the invention, the human gene COL1A2 is Collagen, type I, alpha 2 gene, whose RNA has RNA GenBank accession number NM 000089.

According to the invention, the human gene LGALS4 is Lectin, galactoside-binding, soluble, 4 gene, whose RNA has RNA GenBank accession number NM_006149. According to the invention, the human gene CEACAM6 is Carcinoembryonic antigen-related adhesion molecule 6 gene, whose RNA has RNA GenBank accession number NM_002483.

According to the invention, the sample of the method of the present invention can be a sample of entire blood or of any fraction of blood comprising nucleic acids, such as serum and/or plasma.

According to the present invention, the expression values for each of the analysed genes can be obtained by quantifying the mRNA for each gene in the sample of interest and by comparing these values with those obtained in healthy control samples or with a threshold value calculated previously for each gene.

The control sample taken from a healthy individual can be a sample of total mRNA or RNA, or can also be a pool of mRNA or cDNA comprising, respectively, mRNA or cDNA of interest obtained from healthy individuals. Alternatively, when the expression is quantified with respect to a threshold value predetermined for each RNA, this threshold value will be a threshold value that was determined in advance, for each of the RNAs of interest, from healthy samples or fractions thereof taken from healthy individuals.

For the purposes of the present invention, the quantitative analysis of the mRNAs may be carried out by using any one method of quantitative mRNA analysis known to a person skilled in the art, such as Real Time quantitative PCR, or digital PCR or ultra deep-sequencing.

In a preferred embodiment, the quantitative analysis at point b. is carried out by Real Time PCR, which is a technique well-known to a person skilled in the art.

Real Time PCR, also referred to as quantitative PCR or Real Time quantitative PCR (rtq-PCR), is a method of simultaneous DNA amplification (polymerase chain reaction, or PCR) and quantification.

As known to a person skilled in the art, RNA or mRNA extraction makes it possible to create cDNA by reverse-transcription PCR, which may be amplified by DNA-polymerase chain reaction and quantified after each amplification cycle. Common quantification methods include the use of fluorescent dyes that intercalate with the double-strand (ds) DNA and modified DNA oligonucleotides (referred to as probes) that fluoresce when hybridised with a DNA. Therefore, by Real Time PCR it is possible to measure the relative expression of a gene at a specific time, either in a cell or in a specific tissue type. The combination of these two techniques is often referred to as quantitative RT-PCR.

As known to a person skilled in the art, by means of Real Time PCR an absolute quantification of the concentration of specific RNAs can be carried out by producing a standard calibration curve, or, alternatively, a relative quantification can be carried out by comparing their amount to that of a control gene.

Absolute quantification can use standard samples (plasmid DNA or other DNA forms) whose absolute concentration is known. It must be certain, however, that PCR efficiency be the same for known samples and unknown ones. The relative quantification method is simpler, as it requires the quantification of human control or housekeeping genes to normalise the expression of the studied gene.

The primers for Real Time PCR can be easily designed by the person skilled in the art by suitable programs available to the public, even on the Internet, since the sequences of the markers of the present invention are available on the RNA GenBank and the accession numbers for each marker are provided in the present description.

By way of a mere example, in no way binding for the implementation of the present invention, herein primer pairs for Real Time PCR are provided, suitable for the implementation of the method described herein.

The primer pairs may be designed so as to be used in a single reaction, as they function under the same PCR conditions and do not form aspecific amplificates.

Primer pairs for Real Time PCR specific for sequences of interest are commercially available (for example Sigma Aldrich).

According to an exemplary and non-limiting embodiment of the present invention, Real Time PCR can be carried out using primers of SEQ ID 1 and 2 for TSPAN8; primers of SEQ ID 3 and 4 for COL1A2, primers of SEQ ID 5 and 6 for LGALS4 and primers of SEQ ID 7 and 8 for CEACAM6.

Evidently, the sequences of the mRNAs to be quantified being known, the person skilled in the art could easily design other suitable primer pairs that may be used in a single Real Time PCR reaction and which do not produce aspecific amplificates. The primer pairs will have the chemical modifications commonly used for Real Time PCR primers.

In a preferred embodiment, the method of the present invention also provides the amplification and the quantification, by the same Real Time PCR, of one or more human control housekeeping genes for the normalisation of the values obtained for the markers of interest.

For instance, the human gene B2M, of beta 2 microglobulin, whose RNA GenBank accession number is NM 004048, can be used as control gene.

A non-limiting example of primer for the quantification of a control gene is given by the primers of SEQ ID 9 and 10, which enable gene B2M quantification. Evidently, a person skilled in the art could easily design other primers for the quantification of B2M, or of any other constitutively expressed human gene to be used as control.

For the purposes of the present invention, the term primer has the meaning commonly used in the literature, therefore indicating an oligonucleotide of a length normally ranging from 9 to 50 nucleotides, normally from 15 to 30 nucleotides, with a sequence enabling it to specifically and efficiently hybridise to the sequence of interest, neglecting the aspecific ones.

Evidently, in the method of the present invention, any mRNA of human housekeeping gene to be used as control for the normalisation of the values obtained for the mRNAs of the genes TSPAN8, LGALS4, CEACAM6, COL1A2 could be amplified.

As already mentioned, normalisation could be carried out with respect to one or more control genes.

Therefore, the normalisation will make it possible to calculate a normalised Ct value, denoted in the present description as ΔCt, having calculated for each sample analysed the CT of each marker of interest (TSPAN8, LGALS4, CEACAM6, COL1A2), normalising it with respect to the CT of a constitutively expressed (housekeeping) gene with the following operation:

ΔCt_{marker of interest}=CT_{marker of interest}−CT_{constitutive gene}

therefore, by way of example,

ΔCt_{TSPAN8}=CT_{TSPAN8}−CT_{B2M}

According to the present invention, therefore, the quantitative analysis of the method comprises the calculation of the Ct for each marker and the calculation of the ΔCt normalised with respect to a constitutively expressed gene for each marker. We examined the expression pf the CELTiC panel in the blood of 101 individuals who tested positive in the faecal immunochemical test (FIT test) and who were subjected to colonoscopy. The clinical data obtained are shown in Table 1.

TABLE 1
Clinical information relating to the subjects positive to the faecal
immunochemical test (FIT) participating in the study

| | | | | | Sub- | | | | | Sub- | | | | |
|---|---|---|---|---|---|---|---|---|---|---|---|---|---|---|
| Category | No. | age | M | F | category | No. | age | M | F | category | No. | age | M | F |
| No lesion | 36 | 60 ± 6.4 | 10 | 26 | | | | | | | | | | |
| | | | | | No | 14 | | 2 | 12 | No CCR risk | 22 | | 8 | 14 |
| | | | | | clinical | | | | | | | | | |
| | | | | | evidence | | | | | | | | | |
| | | | | | | | | | | haemorrhoids | 7 | | | |
| | | | | | | | | | | diverticulitis | 13 | | | |
| | | | | | | | | | | aphthoid | 1 | | | |
| | | | | | | | | | | lesion | | | | |
| | | | | | | | | | | angiodysplasia | 1 | | | |
| Polyps | 61 | | 31 | 30 | | | | | | | | | | |
| | | | | | at low | 36 | 62.2 ± 6.7 | 17 | 19 | at high risk | 25 | 60.04 ± 10.5 | 14 | 11 |
| | | | | | risk | | | | | | | | | |

Number

| | | |
|---|---|---|
| 1 | 16 | 8 |
| >1 | 18 | 17 |
| N.D. | 2 | |

dimensions

| | | |
|---|---|---|
| ≤4 mm | 16 | 2 |
| >4 mm ≤10 | 13 | 13 |
| >10 | 0 | 10 |
| N.D. | 7 | 0 |

Type

| | | |
|---|---|---|
| sessile | 28 | 12 |
| pedunculated | 3 | 12 |
| N.S. | 5 | 1 |

histotype

| | | |
|---|---|---|
| serrated | 9 | 6 |
| adenomatous | 29 | 24 |
| hyperplastic | 3 | 1 |
| villous | 4 | 16 |
| N.S. | 4 | 0 |

Position

| | | | | | | |
|---|---|---|---|---|---|---|
| right | | | | | 15 | 9 |
| left | | | | | 12 | 13 |
| rectum | | | | | 4 | 2 |
| N.S. | | | | | 5 | 1 |
| Cancer | 4 | 65 ± 3.5 | 3 | 1 | | |
| G1 | 1 | | | 1 | | |
| G2 | 3 | | 3 | | | |

N.D. = not determined; N.S. = not specified; at low risk = polyp <10 mm in the larger dimension or with histological characteristics of villosity <25%; at high risk = high level of dysplasia or with histological characteristics of villosity >25% or >10 mm in the larger dimension; CRC = colorectal carcinoma; G1 = grade 1; G2 = grade 2.

The colonoscopy made it possible to stratify the cases into various groups (FIG. 1, study plan). The cases analysed in a previous study (Rodia 2016, cited above) were used as reference (67 healthy subjects) or were combined in a group of cases with high-risk lesions or affected by colorectal carcinoma (63 subjects with colorectal carcinoma (FIG. 1, study plan). The expression data obtained is shown in Table 2 below.

TABLE 2
Statistical description

| | N | NFIT | LR | HR/CRC | Total | | | | | | |
|---|---|---|---|---|---|---|---|---|---|---|---|
| No. of subjects | | | | | | Kruskal-Wallis Rank Sum Test (p values) | | | | | |
| | 67 | 36 | 36 | 92 | 231 | N vs | N vs | N vs | NFIT | NFIT vs | LR vs |
| | ΔCt ± SD | ΔCt ± SD | ΔCt ± SD | ΔCt ± SD | ΔCt ± SD | NFIT | LR | HR/CRC | vs LR | HR/CRC | HR/CRC |

CEACAM6

| | | | | | | | | | | | |
|---|---|---|---|---|---|---|---|---|---|---|---|
| mean ± sd | 12.3 ± 1.9 | 14.2 ± 1.1 | 13.6 ± 1.2 | 13.3 ± 1.2 | 13.2 ± 1.6 | <0.001 | 0.004 | 0.005 | 0.116 | <0.001 | 1.091 |
| min | 7.6 | 11.5 | 11.4 | 10.6 | 7.6 | | | | | | |
| max | 15.6 | 16.2 | 15.3 | 16.6 | 16.6 | | | | | | |
| median | 12.6 | 14.4 | 13.7 | 13.4 | 13.4 | | | | | | |

LGALS4

| | | | | | | | | | | | |
|---|---|---|---|---|---|---|---|---|---|---|---|
| mean ± sd | 12.9 ± 2.0 | 15.7 ± 1.3 | 15.3 ± 0.8 | 14.7 ± 1.3 | 14.4 ± 1.8 | <0.001 | <0.001 | <0.001 | 1.4 | 0.003 | 0.034 |
| min | 6.8 | 13.8 | 14 | 10.3 | 6.8 | | | | | | |
| max | 16.4 | 19.5 | 17.5 | 18.3 | 19.5 | | | | | | |
| median | 13.1 | 15.4 | 15.1 | 14.7 | 14.6 | | | | | | |

TSPAN8

| | | | | | | | | | | | |
|---|---|---|---|---|---|---|---|---|---|---|---|
| mean ± sd | 11.3 ± 1.7 | 10.0 ± 1.2 | 9.9 ± 1.4 | 9.6 ± 1.9 | 10.2 ± 1.8 | <0.001 | <0.001 | <0.001 | 3.82 | 1.339 | 1.505 |
| min | 8.3 | 8.2 | 7.4 | 4.8 | 4.8 | | | | | | |
| max | 17.6 | 12.3 | 13.1 | 13.8 | 17.6 | | | | | | |
| median | 11 | 9.8 | 10.1 | 10 | 10.2 | | | | | | |

COL1A2

| | | | | | | | | | | | |
|---|---|---|---|---|---|---|---|---|---|---|---|
| mean ± sd | 11.4 ± 1.9 | 9.7 ± 1.3 | 9.7 ± 1.4 | 9.6 ± 2.0 | 10.2 ± 2.0 | <0.001 | <0.001 | <0.001 | 3.964 | 3.747 | 3.646 |
| min | 7.8 | 7.1 | 6.6 | 4.8 | 4.8 | | | | | | |
| max | 18.2 | 11.8 | 12.8 | 14 | 18.2 | | | | | | |
| median | 11.2 | 9.6 | 9.7 | 9.8 | 10 | | | | | | |
| % male age | 52.2 | 27.8 | 47.2 | 52.2 | | | | | | | |
| mean ± sd | 64.9 ± 14.7 | 60.0 ± 6.4 | 62.2 ± 6.7 | 67.1 ± 11.6 | 65.0 ± 11.5 | 0.176 | 0.736 | 2.555 | 0.527 | 0.006 | 0.222 |

N = healthy individuals;
NFIT = negative colonoscopy;
LR = individuals affected by low-risk lesions;
HR/CRC = individuals with high-risk lesions or CRC

Table 2 shows the values observed for the 4 markers for the healthy individuals (N), for the individuals negative to colonoscopy, but positive to the FIT test (NFIT), for the individuals with low-risk lesions (LR), and for the individuals with high-risk lesions or affected by CRC (HR/CRC). By means of the Kruskal-Wallis test, comparisons were performed between N vs NFIT, N vs LR, N vs HR/CRC, NFIT vs LR, NFIT vs LR, NFIT vs HR/CRC, LR vs HR/CRC and the statistical significance was calculated by means of the value P.

As shown, all 4 markers show significant differences in the comparisons N vs NFIT, N vs LR, N vs HR/CRC, NFIT vs LR.

As can be seen from Table 2, a fine differentiation can be seen for the expression values of the 4 markers in the various groups under consideration. Comparing the expression values obtained for the CELTIC panel on the ROC curves (FIG. 2), very promising values for the AUC, sensitivity and specificity are obtained. More specifically, the comparison N vs LR (FIG. 2, graph in the top-left corner) gives AUC 0.91; sensitivity 79%; specificity 94%. In the comparison N vs NFIT (FIG. 2, graph in the bottom-left corner) AUC 0.93; sensitivity 82%; specificity 97%. This is of great interest not only from a diagnostic point of view, but also from a prognostic point of view: being able to identify low-risk lesions and false positives in the FIT test greatly increases the level of efficiency of the screening and is the current objective of the research on tumour markers in the diagnosis of colorectal carcinoma.

The sensitivity and specificity values are obtained from a graph (ROC curve) representing an area (AUC) whose best value is 1 (area of a perfect square with ordinate and abscissa coinciding with value 100).

Climbing up from the area, and more precisely from the topmost, leftwise-projecting point (the area at issue is more or less “stepped” on the left side of the graph) the specificity percentage (on the abscissa) and the sensitivity percentage (on the ordinate) are determined. The more the area tends to a square with value 1, the higher are the specificity and sensitivity percentages.

The sensitivity and specificity values hardly go in the same direction; often, in fact, high specificity (ability to correctly identify healthy subjects) values are related with low sensitivity (ability to correctly identify diseased subjects) values.

For instance, the Faecal Occult Blood Test (FOBT) commonly used for colorectal cancer diagnosis, though being highly specific (in fact, it is able to identify even very small blood traces present in the faeces), is not very sensitive, as it does not enable to discriminate the cases in which bleeding occurs due to reasons independent from the presence of a tumour, so much that in a subsequent colonoscopy inspection only ⅓ of FOBT-positive cases are found to be diseased.

Evidently, therefore, a test with high specificity and sensitivity is needed in the present state of the art, since a test meeting these requirements has an economical value added (as it enables to proceed with a colonoscopy only on individuals actually having a very high probability of having a colorectal cancer), but, above all, an enormous psychological value, as it enables to rule out false positives with higher accuracy, avoiding the entailed psychological repercussions on the same individuals.

As to the faecal occult blood test (FOBT), generally, the sensitivity of a single FOBT test is deemed to range from 10 to 40%, and only by carrying out 3 sample collections for 3 consecutive days the sensitivity might be brought even to 92%, whereas the specificity is anyhow of 90%. FOBT-positive patients subsequently undergo a more in-depth examination, which is colonoscopy, with a 90% sensitivity and 100% specificity.

Only one-third of FOBT-positive patients prove to be ill with colorectal carcinoma, and the diagnosis is made by colonoscopy. Given the specificity and sensitivity of the method of the invention in distinguishing the various patient classes, the use of the method could substitute the current faecal test, but also, and especially, colonoscopy, which is an invasive and particularly costly test.

The invention also relates to the use of a kit comprising one or more aliquots of reagents for the quantitative analysis of the expression of human genes TSPAN8, LGALS4, CEACAM6, COL1A2 and optionally for a control constitutively expressed human gene, wherein said reagents can be separated for each gene or can be unified for one or more genes for the identification from a sample of human entire blood and/or blood fractions comprising nucleic acids patients at low risk and high risk of developing colorectal cancer and, optionally, patients that are negative to colonoscopy but positive to the analysis for occult blood in the faeces FIT (NFIT).

As mentioned above, the quantitative analysis of gene expression is based on the quantification of the mRNA of the genes of interest.

According to the invention, the human gene TSPAN8 is gene Tetraspanin 8, whose RNA has RNA GenBank accession number NM_004616.

According to the invention, the human gene COL1A2 is Collagen, type I, alpha 2 gene, whose RNA has RNA GenBank accession number NM_000089.

According to the invention, the human gene LGALS4 is Lectin, galactoside-binding, soluble, 4 gene, whose RNA has RNA GenBank accession number NM_006149.

According to the invention, the human gene CEACAM6 is Carcinoembryonic antigen-related adhesion molecule 6 gene, whose RNA has RNA GenBank accession number NM_002483.

According to the invention, the sample for the kit of the present invention can be a sample of can be a sample of entire blood or of any fraction of blood comprising nucleic acids, such as serum and/or plasma.

The reagents for the quantitative analysis for the purposes of the present invention are those commonly used by a person skilled in the art for nucleic acid quantitative analysis methodologies, such as Real Time quantitative PCR, or digital PCR or ultra deep-sequencing, without limiting the present invention thereto.

The reagents specific for the analysis of each mRNA of interest could be provided in one or more aliquots distinct for each mRNA of interest or could be provided in one or more aliquots containing reagents for the quantification of one or more mRNA of interest.

According to one embodiment of the invention, the reagents for the quantitative analysis of the expression of the above-mentioned genes are Real Time PCR primers selectively specific for each of said genes.

The sequence of the mRNAs of the genes of interest being known, the technician in the field could design with extreme ease Real Time PCR primers which enable a selective quantification of the mRNAs of interest. Such primers may also be obtained from commercial sources specialised in the preparation of reagents for Real Time PCR.

According to one exemplary and non-limiting embodiment of the present invention, said primers are the primers of SEQ ID 1 and 2 for TSPAN8; the primers of SEQ ID 3 and 4 for COL1A2, the primers of SEQ ID 5 and 6 for LGALS4 and the primers of SEQ ID 7 and 8 for CEACAM6.

Furthermore, the kit according to any one of the embodiments described herein could contain reagents for quantifying the expression of one or more mRNAs of constitutively expressed (housekeeping) human genes, the expression values obtained could be used to normalise the expression values recorded for the above-described mRNAs of interest, i.e., the mRNAs of the human genes TSPAN8, LGALS4, CEACAM6, COL1A2.

Such reagents could be used to quantify, as already described above in connection with the method, one or more mRNAs of constitutive genes, enabling the normalisation of the values measured for the mRNAs of the genes of interest according to the above-described equation:

ΔCt_{marker of interest}=CT_{marker of interest}−CT_{constitutive gene}

By way of example, in no way to be construed as limitative of the present invention, such reagents may be Real Time PCR primers for one or more constitutive human genes, such as the B2M gene for which, for example, the primers of SEQ ID 9 and 10 may be used.

Moreover, the kit according to any one embodiment of the present invention could further comprise one or more reagents for total RNA or mRNA extraction and/or also reagents for reverse transcription of total RNA.

Furthermore, the kit according to any one embodiment of the invention could further comprise one or more aliquots of Total RNA or mRNA or cDNA of healthy individuals as negative controls and/or of individuals affected by colorectal cancer as positive controls.

### Examples

The following examples aim to illustrate the invention without absolutely being limitative thereof.

The studies which led to the present invention were approved by the Ethics Committee of the “Sant'Orsola-Malpighi” Hospital of Bologna, Italy, and meet the requirements of the Helsinki Declaration of Ethical Principles for medical research involving human subjects.

All subjects involved signed an informed consent form before the start of the studies.

RNA Extraction

Entire blood, put in a tube containing EDTA, was treated for lysis within one hour from collection, by adding the reagent TRIzol LS (Invitrogen, Carlsbad, Calif., USA) and total RNA was extracted according to the provider's protocol. Total RNA extracted from 1 ml of blood was subjected to precipitation with standard ethanol, and the pellet was dissolved in 15 μl of RNAse-free water at a final concentration of up to 0.5 μg/μl and stored at −20° C.

The concentration of all samples of total RNA was quantified with a Nanodrop ND-2000 spectrophotometer (Thermo Fisher Scientific, Waltham, Mass.).

qRT-PCR

300 ng of RNA were subjected to reverse transcriptase with the RevertAid First Strand cDNA Synthesis kit (Carlo Erba Reagents, Milan, Italy) and amplified by using the EvaGreen system (Bio-Rad, Hercules, Calif., USA), according to the provider's instruction. The list of the primers used for the candidate markers and the reference genes is reported in Table 3 below (SIGMA ALDRICH, Milan, Italy).

TABLE 3
The preferred markers were selected on a total of 38′104 loci.

| | | RNA | | |
|---|---|---|---|---|
| Gene | | GenBank | | |
| symbol | Gene name | accession no. | sequence | SEQ ID |
| TSPAN8 | Tetraspanin 8 | NM_004616 | gctgcatgcttctgttgtttt | SEQ ID 1 |
| | | | aacacaattatggcttcctg | SEQ ID 2 |
| COL1A2 | Collagen, type I, | NM_000089 | gtggttactactggattgac | SEQ ID 3 |
| | alpha 2 | | ctgccagcattgatagtttc | SEQ ID 4 |
| LGALS4 | Lecing, | NM_006149 | ttaccctggtcccggacatt | SEQ ID 5 |
| | galactoside- | | agcctcccgaaatatggcac | SEQ ID 6 |
| | binding, | | | |
| | soluble, 4 | | | |
| CEACAM6 | Carcinoembryonic | NM_002483 | cacagtctctggaagtgctcc | SEQ ID 7 |
| | antigen-related | | ggccagcactccaatcgt | SEQ ID 8 |
| | adhesion | | | |
| | molecule 6 | | | |
| B2M | Beta-2- | NM_004048 | tgcctgccgtgtgaaccatgt | SEQ ID 9 |
| | microblobulin | | tgcggcatcttcaaacctccatga | SEQ ID 10 |

Forward primer above, reverse primer below.

The Real Time PCR reactions were carried out with the CFX96 instrument (Bio-Rad, Hercules, Calif.), in duplicate, at 95° C. for 10 min, followed by 40 cycles at 95° C. for 15 sec and 60° C. for 1 min, with melting curve analysis. Each qPCR run always included a negative control without cDNA, and a positive control of cDNA derived from cell line HT-29, in which it is known that the genes of interest are present. The reaction efficiency (E) was calculated from the slope of the standard curve generated with 10-fold serial dilutions of the calibration cDNA according to the formula: E=[10 (−1/slope)−1]×100.

Statistical Analysis

Student's test was adopted to compare the expression levels analysed between the CRC cases and the controls. ROC (Receiving Operating Characteristic) curve analysis was used to assess the accuracy with which the parameters diagnosed CRC, for the purpose of discriminating between CRC patients and controls. Calculations both of the area above the curve and of the intervals corresponding to a 95% confidence were assessed by using Medcalc version 14 for statistical analyses. In order to determine the markers cutoff enabling the best discrimination between the two groups, the discriminating analysis was carried out by using the statistical program SPSS, version 22, as described in Wang H, Zhang X, Wang L, Zheng G, Du L, Yang Y, et al. Investigation of cell free BIRCS mRNA as a serum diagnostic and prognostic biomarker for colorectal cancer. Journal of surgical oncology. 2014; 109(6):574-9. The sets of healthy individuals and of CRC patients were considered as a grouping variable, and the four independent markers were grouped together as foreseen variable.

Quantitative Analysis of mRNA Markers in Blood

Each RNA sample (patients or healthy subjects) was assayed for quality and for expression of the candidate markers listed in Table 3 by quantitative PCR, and the values were normalised with the housekeeping gene B2M. Assayed genes exhibited a single peak in the analysis of the melting curve, and all negative controls yielded no detectable amplification values, corroborating amplification specificity. Expression levels of normalised mRNAs indicated as Delta CT (cutoff cycle) were calculated, which were, respectively, 11.3±1.7 for TSPAN8; 12.9±2 for LGALS4; 11.4±1.9 for COL1A2 and 12.3±1.9 for CEACAM6 in healthy subjects; 10.2±1.8 for TSPAN8; 14.4±1.8 for LGALS4; 10.2±2 for COL1A2 and 13.2±1.6 for CEACAM6 in patients affected by CRC or with high-risk lesions; 13.6±1.2; for CEACAM6; 15.3±0.8 for LGALS4; 9.9±1.4 for TSPAN8; and Delta Ct 9.7±1.4 for COL1A2 in patients with low-risk lesions, and 14.2±1.1 for CEACAM6; 15.7±1.3 for LGALS4; 10.0±1.2 for TSPAN8 and 9.7±1.3 for COL1A2 in patients negative to colonoscopy, but positive to the analysis for occult blood in the FIT (NFIT).

Diagnostic Value of mRNA Markers in Blood for CRC

In order to assess diagnostic accuracy in terms of specificity and sensitivity of candidate markers, ROC curve analysis was carried out. Graphic processing for these four markers is reported in FIG. 2.

Prognostic Method.

1. 2 ml of peripheral blood were collected from patients affected from evident colorectal cancer and from healthy donors who had given their approval to collection with an informed consent.

2. Blood was lysed with Trizol LS following the producer's instructions. Preferably, lysis was carried out within 1-2 hours from collection.

3. Total RNA was extracted from samples according to standard protocols of commercial kits.

4. Retrotranscription of 300 ng of RNA was carried out for each sample.

5. Real Time PCR reactions were carried out by using the primers reported in Table 2, for all combinations reported in Table 1, comprising primers for a housekeeping gene as control for data normalisation.

6. Data analysis was carried out by normalising the values measured for the mRNAs of the genes of interest according to the equation:

ΔCt_{marker of interest}=CT_{marker of interest}−CT_{constitutive gene}

The analysis thus carried out yielded the data reported in Table 2.

### CONCLUSIONS

Hence, the prognostic method of the invention enables a screening on entire blood that can not only facilitate an early diagnosis of CRCm but also makes it possible to identify patients at low risk and high risk of developing colorectal cancer. It should be stressed that the use of entire blood enables the detection of mRNA molecules present in CRC patients' blood of with an expression altered with respect to normal individuals. Collected blood is preferably lysed with Trizol LS within 1 hour from collection in order to avoid possible degradation of said molecules.

The quantitative analysis of the panel of the 4 markers reveals very promising sensitivity and specificity values of around, respectively 79 and 94% when the comparison is between healthy subjects and subjects with low-risk lesions, and of 75 and 87% when the comparison is between healthy subjects and subjects at high risk of colorectal carcinoma. The method also demonstrated such selectivity with respect to the evidencing of false positives of the FIT test, with sensitivity of 82% and specificity of 97%, so as to be comparable to the sensitivity and specificity values of colonoscopy.

What is claimed is:

1. A method of treatment for patients at risk of developing colorectal cancer (CRC) wherein before medical treatment, risk of developing colorectal cancer is assessed by analysing a sample of blood and/or blood fractions comprising nucleic acids of said patients, comprising the steps of:
a. extracting total RNA or mRNA from said sample;
b. quantitatively analyzing human TSPAN8, LGALS4, CEACAM6, and COL1A2 mRNAs;
c. quantifying the mRNA of one or more human constitutive housekeeping genes for the normalization of the results;
d. assessing the risk that the subject of the analysed sample has to develop colorectal cancer, wherein
for a value of ΔCt 13.6±1.2 for CEACAM6; a value of ΔCt 15.3±0.8 for LGALS4; a value of ΔCt 9.9±1.4 for TSPAN8; and a value of ΔCt 9.7±1.4 for COL1A2 the subject analysed is defined as a patient with lesions at low risk for developing colorectal cancer and,
e. the patients with lesions at low risk for developing colorectal cancer are subject to routine long term evaluation;
and subsequently administering the medical treatment.

2. The method according to claim 1, further comprising the identification of subjects that are negative to colonoscopy but positive to the analysis for occult blood in the faeces immunochemical test (FIT) wherein,
for a value of ΔCt 14.2±1.1 for CEACAM6; a value of ΔCt 15.7±1.3 LGALS4; a value of ΔCt 10.0±1.2 for TSPAN8 and a value of ΔCt 9.7±1.3 for COL1A2, the subject analysed is defined as a patient negative to colonoscopy but positive to the analysis for occult blood in the faeces FIT.

3. The method according to claim 1, wherein the quantitative analysis comprises Real Time PCR.

4. The method according to claim 3, wherein the Real Time PCR is carried out using primers of SEQ ID: 1 and SEQ ID: 2 for TSPAN8, primers of SEQ ID: 3 and SEQ ID: 4 for COL1A2, primers of SEQ ID: 5 and SEQ ID: 6 for LGALS4, and primers of SEQ ID: 7 and SEQ ID: 8 for CEACAM6.

5. A method of treatment for patients at risk of developing colorectal cancer (CRC) wherein before medical treatment, risk of developing colorectal cancer is assessed by analysing a sample of blood and/or blood fractions comprising nucleic acids of said patients, comprising the steps of:
a. extracting total RNA or mRNA from said sample;
b. quantitatively analyzing human TSPAN8, LGALS4, CEACAM6, and COL1A2 mRNAs;
c. quantifying the mRNA of one or more human constitutive housekeeping genes for the normalization of the results;
d. assessing the risk that the subject of the analysed sample has to develop colorectal cancer, wherein
for a value of ΔCt 9.6±1.9 for TSPAN8; a value of ΔCt 9.6±2 for COL1A2; a value of ΔCt 14.7±1.3 for LGALS4, and a value of ΔCt 13.3±1.2 for CEACAM6, the subject analysed is defined as a patient with lesions at high risk of developing colorectal cancer or affected by CRC; and
e. patients with lesions at high risk of developing colorectal cancer or affected by CRC are subjected to medical surgery for removing said lesions;
and subsequently administering the medical treatment.

6. The method according to claim 5, further comprising the patients are subjected to ongoing short-term evaluation.

7. The method according to claim 6, further comprising the patients are subjected to therapy with anti-cancer drugs.

8. The method according to claim 7, further comprising the identification of subjects that are negative to colonoscopy but positive to the analysis for occult blood in the faeces immunochemical test (FIT) wherein,
for a value of ΔCt 14.2±1.1 for CEACAM6; a value of ΔCt 15.7±1.3 LGALS4; a value of ΔCt 10.0±1.2 for TSPAN8 and a value of ΔCt 9.7±1.3 for COL1A2, the subject analysed is defined as a patient negative to colonoscopy but positive to the analysis for occult blood in the faeces FIT.

9. The method according to claim 7, wherein the quantitative analysis comprises Real Time PCR.

10. The method according to claim 9, wherein the Real Time PCR is carried out using primers of SEQ ID: 1 and SEQ ID: 2 for TSPAN8, primers of SEQ ID: 3 and SEQ ID: 4 for COL1A2, primers of SEQ ID: 5 and SEQ ID: 6 for LGALS4, and primers of SEQ ID: 7 and SEQ ID: 8 for CEACAM6.

11. A kit comprising nucleic acids, one or more aliquots of reagents for the quantitative analysis of the expression of human genes TSPAN8, LGALS4, CEACAM6, COLIA2, and optionally for a control constitutively expressed human gene, wherein said reagents can be separated for each gene or can be unified for one or more genes for the identification from a sample of human entire blood and/or blood fractions from patients at risk of developing colorectal cancer and, optionally, patients that are negative to colonoscopy but positive to the analysis for occult blood in the faeces FIT.

12. The kit according to claim 11, wherein the reagents are Real Time PCR reagents that are selectively specific for each of said genes.

13. The kit according to claim 11, wherein the reagents comprise primers of SEQ ID: 1 and SEQ ID: 2 for TSPAN8, primers of SEQ ID: 3 and SEQ ID NO: 4 for COL1A2, primers of SEQ ID: 5 and SEQ ID NO: 6 for LGALS4, and primers of SEQ ID NO: 7 and SEQ ID NO: 8 for CEACAM6.

14. The kit according to claim 11, further comprising Real Time PCR primers for a mRNA of one or more constitutive (housekeeping) human genes.

15. The kit according to claim 11, further comprising reagents for total RNA or mRNA extraction.

16. The kit according to claim 11, further comprising one or more aliquots of total RNA, mRNA, or cDNA from healthy individuals as negative controls and/or from individuals affected by colorectal cancer as positive controls.

17. A method of identifying colorectal tumor cells in a sample comprising:
a. extracting total RNA or mRNA from said sample;
b. quantitatively analyzing human TSPAN8, LGALS4, CEACAM6, and COL1A2 mRNAs;
c. quantifying the mRNA of one or more human constitutive housekeeping genes for the normalization of the results;
d. assessing the risk that the subject of the analysed sample has to develop colorectal cancer, wherein
for a value of ΔCt 13.6±1.2 for CEACAM6; a value of ΔCt 15.3±0.8 for LGALS4; a value of ΔCt 9.9±1.4 for TSPAN8; and a value of ΔCt 9.7±1.4 for COL1A2 the subject analysed is defined as a patient with lesions at low risk for developing colorectal cancer and,
e. the patients with lesions at low risk for developing colorectal cancer are subject to routine long term evaluation.

18. A method of identifying colorectal tumor cells in a sample comprising:
a. extracting total RNA or mRNA from said sample;
b. quantitatively analyzing human TSPAN8, LGALS4, CEACAM6, and COL1A2 mRNAs;
c. quantifying the mRNA of one or more human constitutive housekeeping genes for the normalization of the results;
d. assessing the risk that the subject of the analysed sample has to develop colorectal cancer, wherein
for a value of ΔCt 9.6±1.9 for TSPAN8; a value of ΔCt 9.6±2 for COL1A2; a value of ΔCt 14.7±1.3 for LGALS4, and a value of ΔCt 13.3±1.2 for CEACAM6, the subject analysed is defined as a patient with lesions at high risk of developing colorectal cancer or affected by CRC; and
e. patients with lesions at high risk of developing colorectal cancer or affected by CRC are subjected to medical surgery for removing said lesions.
